(19) United States
(12) Patent Application Publication
(10) Pub. No.: US 2011/0046229 A1
(43) Pub. Date: Feb. 24, 2011

(54) Controlled Release Drug Delivery Systems And Pharmaceutical Compositions Formed Therewith

(75) Inventor: Elena S. Draganoiu, Sagamore Hills, OH (US)

Correspondence Address: THE LUBRIZOL CORPORATION, 29400 LAKELAND BLVD, MAIL DROP 022B, WICKLIFFE, OH, 44092-2298, US

(73) Assignee: LUBRIZOL ADVANCED MATERIALS, INC., Cleveland, OH (US)

(21) Appl. No.: 12/920,562

(22) PCT Filed: Jan. 22, 2009
(86) PCT No.: PCT/US09/31634

§ 371 Date: Sep. 1, 2010

Related U.S. Application Data
(60) Provisional application No. 61/033,929, filed on Mar. 5, 2008.

(51) Int. Cl. A61K 31/167; A61P 43/00

(52) U.S. Cl. 514/629

(57) ABSTRACT

The present invention relates to controlled release drug delivery systems. In one embodiment, the present invention relates to controlled release drug delivery systems comprising a combination of at least one polyacrylic acid and at least one enteric polymer. In another embodiment, the present invention relates to a controlled release pharmaceutical composition comprising the combination of at least one active pharmaceutical ingredient and a controlled release drug delivery system, where the controlled release drug delivery system comprises the combination of at least one polyacrylic acid and at least one enteric polymer.

### FIELD OF THE INVENTION

The present invention relates to controlled release drug delivery systems. In one embodiment, the present invention relates to controlled release drug delivery systems comprising a combination of at least one polyacrylic acid and at least one enteric polymer. In another embodiment, the present invention relates to a controlled release pharmaceutical composition comprising the combination of at least one active pharmaceutical ingredient and a controlled release drug delivery system, where the controlled release drug delivery system comprises the combination of at least one polyacrylic acid and at least one enteric polymer.

### BACKGROUND OF THE INVENTION

Controlled release drug delivery systems are known in the art. Such controlled release systems are important for delivering long-term dosages of various drugs, or combinations of drugs, over extended periods of time (e.g., up to 12 hours, or up to 24 hours).

To date, the majority of controlled release drug delivery systems based on carbomers work better in a region of higher pH (e.g., the intestines). This is due to the fact that gastric fluids generally penetrate a carbomer-based delivery system before the formation of a desired gel matrix. Due to this technical aspect, carbomer-based controlled release drug delivery systems are generally more efficient at achieving the controlled release of active pharmaceutical ingredients (APIs) in a higher pH environment. Accordingly, this makes it difficult to achieve substantially similar release profiles in areas of both low and higher pH given the disparity between the low and higher pH release profiles for a carbomer/API release system. Thus, there is currently a lack in the art of carbomer-based controlled release drug delivery systems that are pH independent and efficiently and/or uniformly deliver a wide range of active pharmaceutical ingredients (APIs).

One attempt at achieving a controlled release drug delivery system formed from the combination of a hydrophilic polymer and an enteric polymer is disclosed in U.S. Pat. No. 6,074,669. However, U.S. Pat. No. 6,074,669 is only concerned with the controlling the release profile of diltiazem or a pharmaceutically acceptable salt, or ester thereof, in a low pH environment (i.e., the stomach), and, as such, utilizes an enteric polymer to combat the pH dependent solubility of diltiazem, which would result in the unacceptably faster release of diltiazem in a low pH environment versus a higher pH environment.

Given the above, there is a need for a controlled release drug delivery system that is not only pH independent, but one that permits delivery of a wide variety of active pharmaceutical ingredients (APIs) regardless of whether or not the drug, or drugs, to be delivered exhibit pH dependency.

### SUMMARY OF THE INVENTION

The present invention relates to controlled release drug delivery systems. In one embodiment, the present invention relates to controlled release drug delivery systems comprising a combination of at least one polyacrylic acid and at least one enteric polymer. In another embodiment, the present invention relates to a controlled release pharmaceutical composition comprising the combination of at least one active pharmaceutical ingredient and a controlled release drug delivery system, where the controlled release drug delivery system comprises the combination of at least one polyacrylic acid and at least one enteric polymer.

In one embodiment, the present invention relates to a controlled release drug delivery system comprising: at least one polyacrylic acid; and at least one enteric polymer, wherein the controlled release drug delivery system is able to deliver in a controlled release manner at least one active pharmaceutical ingredient regardless of whether or not the at least one active pharmaceutical ingredient exhibits pH dependency.

In another embodiment, the present invention relates to controlled release drug delivery system comprising: from about 5 weight percent to about 95 weight percent of at least polyacrylic acid; and from about 95 weight percent to about 5 weight percent of at least one enteric polymer, wherein the controlled release drug delivery system is able to deliver in a controlled release manner at least one active pharmaceutical ingredient regardless of whether or not the at least one active pharmaceutical ingredient exhibits pH dependency.

In still another embodiment, the present invention relates to a controlled release pharmaceutical composition comprising: at least one active pharmaceutical ingredient; and at least one controlled release drug delivery system, wherein the controlled release drug delivery system comprises: at least one polyacrylic acid; and at least one enteric polymer, wherein the controlled release drug delivery system is able to deliver in a controlled release manner the at least one active pharmaceutical ingredient regardless of whether or not the at least one active pharmaceutical ingredient exhibits pH dependency.

In still yet another embodiment, the present invention relates to a controlled release pharmaceutical composition comprising: at least one active pharmaceutical ingredient; and at least one controlled release drug delivery system, wherein the controlled release drug delivery system comprises: from about 5 weight percent to about 95 weight percent of at least polyacrylic acid; and from about 95 weight percent to about 5 weight percent of at least one enteric polymer, wherein the controlled release drug delivery system is able to deliver in a controlled release manner the at least one active pharmaceutical ingredient regardless of whether or not the at least one active pharmaceutical ingredient exhibits pH dependency.

### BRIEF DESCRIPTION OF THE DRAWINGS

FIG. 1 is a graph illustrating the amount, in weight percent, of acetaminophen released up to a given time in simulated gastric fluid (SGF) without pepsin over time. The legend expresses the weight percent of carbomer/enteric polymer;

FIG. 2 is a graph illustrating the amount, in weight percent, of acetaminophen released up to a given time in a pH=6.8 buffer (USP Phosphate Buffer) over time. The legend express the weight percent of carbomer/enteric polymer; and

FIG. 3 is a graph illustrating the differences in the amount of acetaminophen released in simulated gastric fluid (SGF) without pepsin versus a buffer having a pH=6.8 (USP phosphate buffer) at different time intervals. The legend expresses the weight percent of carbomer/enteric polymer.

### DETAILED DESCRIPTION OF THE INVENTION

The present invention relates to controlled release drug delivery systems. In one embodiment, the present invention relates to controlled release drug delivery systems comprising a combination of at least one polyacrylic acid and at least one enteric polymer. In another embodiment, the present invention relates to a controlled release pharmaceutical composition comprising the combination of at least one active pharmaceutical ingredient (API) and a controlled release drug delivery system, where the controlled release drug delivery system comprises the combination of at least one polyacrylic acid and at least one enteric polymer.

As used throughout the specification and claims, the term polyacrylic acid or acrylic acid polymers is used to encompass a variety of polymers having high percentages of polymerizable monomers therein with pendant carboxylic acid groups or anhydrides of polycarboxylic acid. These compounds are described in more detail in U.S. Pat. Nos. 2,798,053; 3,915,921; 4,267,103; 5,288,814; and 5,349,030, all of which are hereby incorporated by reference in there entireties. The term polyacrylic acid is also used to include various homopolymers, copolymers, and interpolymers, wherein at least 50 or 75 mole percent of the repeating units have pendant carboxylic acid groups or anhydrides of dicarboxylic acid groups. While acrylic acid is the most common primary monomer used to form polyacrylic acid the term is not limited thereto but includes generally all α-β unsaturated monomers with carboxylic pendant groups or anhydrides of dicarboxylic acids as described in U.S. Pat. No. 5,349,030.

As is discussed above, in one embodiment the present invention is a controlled release drug delivery systems comprising a combination of at least one polyacrylic acid (e.g., a carbomer) and at least one enteric polymer. It should be noted that the present invention is not limited to any specific enteric polymer. Rather, any suitable enteric polymer can be used so long as the enteric polymer chosen functions properly in combination with both the carbomer portion of the controlled release drug delivery system of the present invention and the one or more APIs to be delivered by such system.

In one embodiment, cross-linked polyacrylic acids can be utilized in the controlled release drug delivery systems of the present invention. Suitable cross-linked polyacrylic acids include, but are not limited to polycarbophils, carbomers, Carbopol® polymers, Carbopol homopolymers, Carbopol copolymers, Carbopol interpolymers (e.g., Carbopol® 971PNF), copolymers of acrylic acid and alkyl acrylates, copolymers of acrylic acid and alkyl vinyl ethers, copolymers of ethylene and maleic anhydride, copolymers of maleic anhydride and alkyl vinyl ethers, or combinations of two or more thereof. An approved polyacrylic acid for pharmaceutical applications, described in a carbomer monograph in the U.S.P. Pharmacopeia 30 NF 25, is a polyacrylic acid crosslinked with polyalkenyl ethers.

In one embodiment, suitable enteric polymers for use in conjunction with the present invention include, but are not limited to, polyacrylate copolymers such as methacrylic acid copolymer, USP/NF, Types A, B or C (which are available from Evonik Industries AG under the brand name Eudragit®); cellulose derivatives (e.g., cellulose acetate phthalate, cellulose acetate trimellitate, hydroxypropyl methylcellulose phthalate, or hydroxypropyl methylcellulose acetate succinates); polyvinyl acetate phthalate; shellac; or suitable combinations of two or more thereof.

In another embodiment, enteric polymers suitable for use in conjunction with the present invention include, but are not limited to, pharmaceutically-acceptable forms of cellulose, vinyl, and acrylic polymer derivatives. These polymers exhibit resistance to gastric fluids yet are readily soluble or permeable in intestinal fluid. Enteric polymeric materials are primarily weak acids containing acidic functional groups, which are capable of ionization at elevated pH (above a pH of about 5). In the low pH of the stomach, enteric polymers remain unionized, and therefore, insoluble. As the pH increases in the intestinal tract, the functional groups present in enteric polymers ionize, and the polymer becomes soluble in the intestinal fluids.

In one embodiment, a controlled release drug delivery system according to the present invention comprises from about 3 weight percent to about 97 weight percent of at least one polyacrylic acid (i.e. a carbomer) and from about 97 weight percent to about 3 weight percent of at least one enteric polymer. In another embodiment, the amount of the polyacrylic acid-based portion of a controlled release drug delivery system according to the present invention is in the range of about 5 weight percent to about 95 weight percent polyacrylic acid, or from about 7.5 weight percent to about 90 weight percent polyacrylic acid, or from about 10 weight percent to about 85 weight percent polyacrylic acid, or from about 15 weight percent to about 80 weight percent polyacrylic acid, or from about 20 weight percent to about 75 weight percent polyacrylic acid, or from about 25 weight percent to about 70 weight percent polyacrylic acid, or even from about 30 weight percent to about 65 weight percent polyacrylic acid, with the remainder of the controlled release drug delivery system of this embodiment being at least one enteric polymer. Here, as well as else where in the specification and claims, individual range limits can be combined to form additional non-disclosed range limits.

In still another embodiment, the amount of the polyacrylic acid-based portion of a controlled release drug delivery system according to the present invention is in the range of about 35 weight percent to about 60 weight percent polyacrylic acid, or from about 40 weight percent to about 55 weight percent polyacrylic acid, or from about 45 weight percent to about 50 weight percent polyacrylic acid, with the remainder of the controlled release drug delivery system of this embodiment being at least one enteric polymer. It should be noted that here, as well as elsewhere in the specification and claims, individual range limits from one embodiment can be combined with range limits from one or more alternative embodiments to form additional non-disclosed embodiments.

In yet another embodiment, the controlled release drug delivery systems of the present invention can optionally further contain pharmaceutically acceptable additives such as diluents, binders, lubricants, glidants, coatings, buffering agents, preservatives, stabilizers, surfactants, colorants, disintegrants, plasticizers, modified release agents, controlled release agents, and the like.

The controlled release drug delivery system of the present invention can be formed into any suitable dosage form. Such forms include, but are not limited to, tablets, pills, capsules, gel-tabs, granules, beads, or an aqueous dispersion of one or more thereof. Depending upon the desired dosage form for the controlled release drug delivery system of the present invention various manufacturing methods can be used. Such methods include, but are not limited to, direct compression, wet granulation, roller compaction, hot-melt granulation, or the like.

Due to the makeup of the controlled release drug delivery systems described above, the present invention permits one to achieve controlled release of one or more APIs regardless of pH. That is, in one instance, the controlled release drug delivery systems of the present invention are not pH dependent and thus permit generally uniform delivery of one or more APIs in both the stomach and intestines of a recipient.

In still yet another embodiment, the present invention is directed to a controlled release pharmaceutical composition comprising the combination of at least one API and a controlled release drug delivery system, where the controlled release drug delivery system comprises the combination of at least one polyacrylic acid and at least one enteric polymer. In this embodiment, the controlled release drug delivery system is as described above.

Turning to the types of APIs for use in this embodiment, such compounds are not restricted to any one class or type of drug. In one embodiment, the present invention permits the controlled release of cationic, anionic, amphoteric, zwitterionic, nonionic, or suitable combinations of two or more thereof.

In this embodiment, the amount of API, or drug, present in the controlled release pharmaceutical compositions of the present invention can be up to about 85 weight percent of a dosage form (e.g., a pill, capsule, tablet, gel-tab, etc.), with the remainder of the dosage form being selected from one of the controlled release drug delivery systems described above. In another embodiment, the amount of API, or drug, present in a dosage form is up to about 80 weight percent, up to about 75 weight percent, up to about 70 weight percent, up to about 65 weight percent, up to about 60 weight percent, up to about 55 weight percent, up to about 50 weight percent, up to about 40 weight percent, up to about 40 weight percent, or even up to about 35 weight percent, with the remainder of the dosage form being selected from one of the controlled release drug delivery systems described above. In still another embodiment, the amount of API, or drug, present in a dosage form is in the range of about 0.5 weight percent to about 85 weight percent, or 5 weight percent to about 75 weight percent, or even 10 weight percent to about 55 weight percent. It should be noted that here, as well as elsewhere in the specification and claims, individual range limits from one embodiment can be combined with range limits from one or more alternative embodiments to form additional non-disclosed embodiments.

In yet another embodiment, the controlled release pharmaceutical compositions of the present invention can optionally further contain pharmaceutically acceptable additives such as diluents, binders, lubricants, glidants, coatings, buffering agents, preservatives, stabilizers, surfactants, colorants, disintegrants, plasticizers, modified release agents, controlled release agents, and the like.

### EXAMPLES

The following examples are for illustrative purposes only. As such, the present invention is not limited thereto, but should be broadly construed.

Examples of combination of a polyacrylic acid (i.e., a carbomer) and enteric polymer aimed to minimize the effect of pH on carbomer-based matrix for the controlled release of acetaminophen, a drug that does not exhibit pH-dependent solubility, are shown in Tables 1 and 2.

TABLE 1
Effect of Polymer Combination on the Difference
of the Amount of Drug Released in Two Media

| | Formulation | | | |
|---|---|---|---|---|
| Carbomer (Weight Percent - | 25 | 0 | 20 | 20 |
| Carbopol ® 71GNF) | | | | |
| Enteric Polymer (Weight | 0 | 25 | 20 | 25 |
| Percent - Eudragit ® L100-55) | | | | |
| | Difference in the Amount | | | |
| Time (Hours) | Released in Two Media | | | |
| 1 | 5.20 | −30.07 | 6.47 | 5.04 |
| 2 | 8.35 | −53.31 | 10.06 | 7.70 |
| 3 | 11.96 | −65.25 | 12.13 | 8.95 |
| 4 | 20.24 | −65.22 | 13.06 | 9.27 |
| 5 | 28.97 | −61.35 | 13.24 | 8.92 |
| 6 | 56.73 | −58.46 | 12.96 | 8.22 |
| 7 | 72.60 | −55.28 | 12.47 | 7.37 |
| 8 | 68.15 | −52.33 | 11.78 | 6.36 |
| 9 | 63.63 | −49.46 | 10.96 | 5.40 |
| 10 | 59.25 | −46.84 | 10.20 | 4.45 |
| 11 | 54.96 | −44.37 | 9.47 | 3.64 |
| 12 | 50.61 | −42.14 | 8.61 | 2.76 |
| 13 | 46.31 | −40.01 | 7.47 | 1.94 |
| 14 | 41.95 | −38.14 | 6.23 | 1.13 |
| 15 | 37.80 | −36.22 | 5.11 | 0.19 |
| 16 | 33.24 | −34.57 | 3.95 | −0.72 |
| 17 | 24.16 | −32.93 | 2.78 | −1.65 |
| 18 | 6.24 | −31.47 | 1.66 | −2.59 |
| 19 | 0.95 | −30.02 | 0.63 | −3.49 |
| 20 | −0.31 | −28.91 | −0.66 | −4.37 |

With regard to the above Examples, the remainder of the composition needed to achieve 100 weight percent is 54.17 weight percent acetaminophen with the remainder comprising fillers and lubricants. In Table 1, the effect of pH is quantified by the difference in the amount of drug released at different time intervals in simulated gastric fluid (SGF) without pepsin versus a buffer having a pH=6.8 (USP phosphate buffer). The ideal is to obtain a difference as close to zero as possible, thereby resulting in pH independent release. Thus, in the 0 weight percent carbomer/25 weight percent enteric polymer far less of the drug is released in the simulated gastric fluid (SGF) without pepsin solution, thereby illustrating the pH dependency of the enteric polymer. Accordingly, the above data illustrates that by using just carbomer or enteric polymer, the effect of pH is very significant, while by using combination the difference is much smaller. By using the combination the resulting release is intermediate to the profiles obtained by using each polymer alone.

TABLE 2
Drug Released in Two Media from Formulations
Containing Carbomer and Enteric Polymer

| | Formulation | | | |
|---|---|---|---|---|
| Carbomer | 20 | | 20 | |
| (Weight Percent - | | | | |
| Carbopol ® | | | | |
| 71GNF) | | | | |
| Enteric polymer | 20 | | 25 | |
| (Weight Percent - | | | | |
| Eudragit ® | | | | |
| L100-55) | | | | |
| | Amount released | | | |
| | | pH = 6.8 (USP | SGF | pH = 6.8 (USP |
| | SGF w/o | Phosphate | w/o | Phosphate |
| Time (Hours) | Pepsin | Buffer) | Pepsin | Buffer) |
| 1 | 10.83 | 4.36 | 8.58 | 3.54 |
| 2 | 18.29 | 8.24 | 14.52 | 6.81 |
| 3 | 24.30 | 12.16 | 19.31 | 10.35 |
| 4 | 29.53 | 16.47 | 23.47 | 14.20 |
| 5 | 34.29 | 21.04 | 27.29 | 18.37 |
| 6 | 38.81 | 25.85 | 30.84 | 22.62 |
| 7 | 43.28 | 30.81 | 34.36 | 26.99 |
| 8 | 47.44 | 35.66 | 37.65 | 31.29 |
| 9 | 51.65 | 40.68 | 40.73 | 35.33 |
| 10 | 55.81 | 45.61 | 43.72 | 39.26 |
| 11 | 59.75 | 50.29 | 46.65 | 43.01 |
| 12 | 63.52 | 54.91 | 49.40 | 46.64 |
| 13 | 66.89 | 59.42 | 52.09 | 50.15 |
| 14 | 70.15 | 63.92 | 54.58 | 53.45 |
| 15 | 73.31 | 68.20 | 57.00 | 56.82 |
| 16 | 76.26 | 72.31 | 59.30 | 60.01 |
| 17 | 79.14 | 76.36 | 61.45 | 63.11 |
| 18 | 81.89 | 80.23 | 63.46 | 66.06 |
| 19 | 84.57 | 83.94 | 65.47 | 68.97 |
| 20 | 87.08 | 87.74 | 67.36 | 71.73 |

With regard to the above Examples, the remainder of the composition needed to achieve 100 weight percent is 54.17 weight percent acetaminophen with the remainder comprising fillers and lubricants. Table 2 shows the amount released in the two media from the two formulations containing polymer combination; closer values in the two media for each formulation indicate less pH effect.

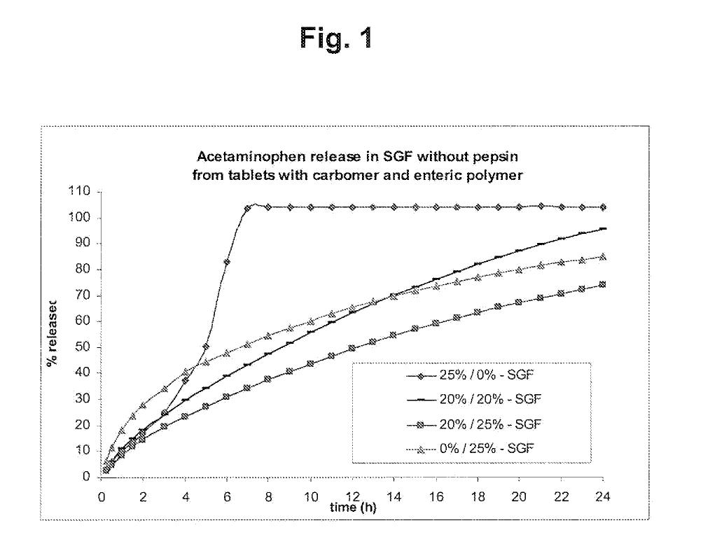

Turning to the Figures, FIGS. 1 through 3 illustrate the amount, in weight percent, of acetaminophen released up to a given time in simulated gastric fluid (SGF) without pepsin over time for various combinations of carbomer and enteric polymer.

Specifically, the graph of FIG. 1 illustrates the amount, in weight percent, of acetaminophen released up to a given time in simulated gastric fluid (SGF) without pepsin over time for the formulations of Table 1 where the amount of carbomer is listed first in the legend of the graph of FIG. 1.

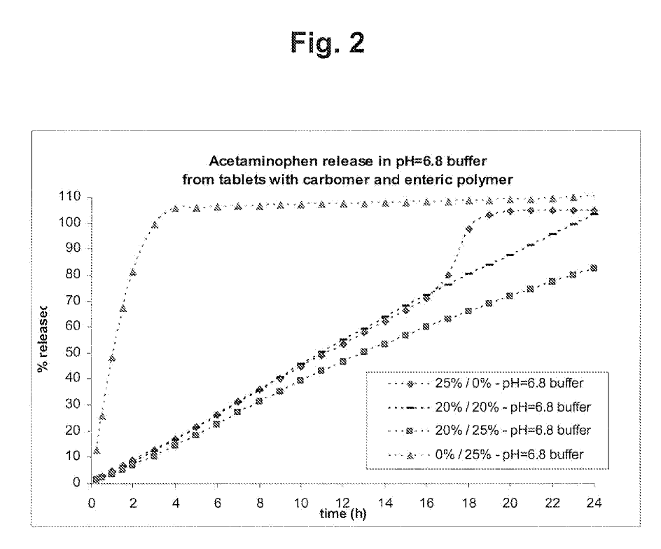

Specifically, the graph of FIG. 2 illustrates the amount, in weight percent, of acetaminophen released up to a given time in a pH=6.8 buffer (USP Phosphate Buffer) over time for the formulations of Table 1 where the amount of carbomer is listed first in the legend of the graph of FIG. 2.

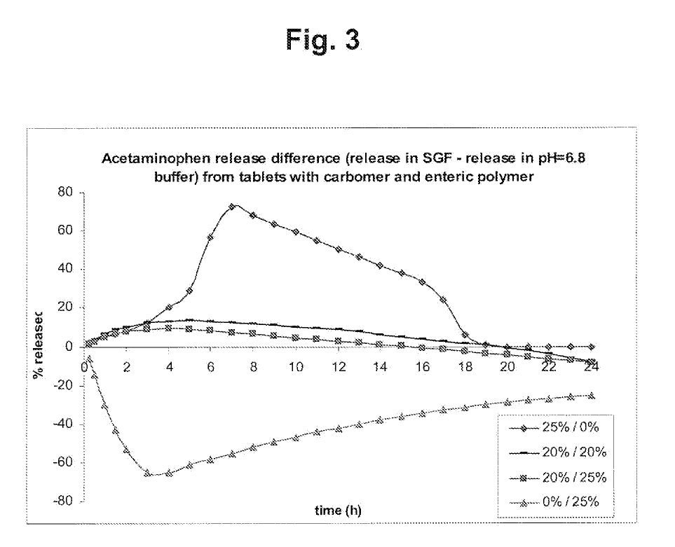

With regard to FIG. 3, the graph of FIG. 3 illustrates the differences in the amount of acetaminophen released in simulated gastric fluid without pepsin versus a buffer having a pH=6.8 (USP phosphate buffer) at different time intervals. Some of the data points from FIG. 3 are represented in table form above in Table 1.

Thus, from the data contains in Tables 1 and 2, as well as the graphs of FIGS. 1 through 3, it can be seen that the controlled release drug delivery systems of the present invention achieve pH independent release of a drug in both “stomach-” and “intestine-type” conditions.

In one embodiment, the controlled release drug delivery systems of the present invention achieve substantially similar release profiles of a wide variety of drugs over a wide pH range. In this instance, the above result is achieved via the combination of at least one polyacrylic acid (e.g., a carbomer) and at least one enteric polymer. This combination is advantageous in that the enteric polymer “protects” the at least one polyacrylic acid in a low pH environment, while also appropriately controlling the release of the one or more APIs.

In light of the above, the controlled release drug delivery systems of the present invention permit the administration of various APIs in controlled release forms to a subject at a constant rate over a wide range of pHs. Accordingly, pH independent delivery of a wide variety of APIs is achieved via the controlled release drug delivery systems of the present invention. Since enteric, or pH-dependent polymers, are insoluble at the acidic pH level found in the stomach but dissolve and/or erode in the higher pH environment found in the intestines (i.e., a pH of about 5 to about 8), the combination of at least one polyacrylic acid (i.e., carbomer) with at least one enteric polymer composition permits delivery at more or less a constant rate of an API in both the stomach and intestines.

While in accordance with the patent statutes the best mode and certain embodiments have been set forth, the scope of the invention is not limited thereto, but rather by the scope of the attached.

1. A controlled release drug delivery system comprising:
at least one polyacrylic acid; and
at least one enteric polymer,
wherein the controlled release drug delivery system is able to deliver in a controlled release manner at least one active pharmaceutical ingredient regardless of whether or not the at least one active pharmaceutical ingredient exhibits pH dependency.

2. The controlled release drug delivery system of claim 1, wherein the at least one enteric polymer is selected from one or more polyacrylate copolymers, one or more cellulose derivatives, one or more polyvinyl acetate phthalates, shellac, or suitable combinations of two or more thereof.

3. The controlled release drug delivery system of claim 1, wherein the at least one polyacrylic acid is a cross-linked polyacrylic acid selected from one or more carbomers, one or more polycarbophils, one or more copolymers of acrylic acid and alkyl acrylates, one or more copolymers of acrylic acid and alkyl vinyl ethers, copolymers of ethylene and maleic anhydride, one or more copolymers of maleic anhydride and alkyl vinyl ethers, or combinations of two or more thereof.

4. The controlled release drug delivery system of claim 1, wherein the delivery system can be used in conjunction with at least one active pharmaceutical ingredient selected from one or more cationic pharmaceutical compounds, one or more anionic pharmaceutical compounds, one or more amphoteric pharmaceutical compounds, one or more zwitterionic pharmaceutical compounds, one or more nonionic pharmaceutical compounds, or suitable combinations of two or more thereof.

5. The controlled release drug delivery system of claim 1, wherein the controlled release drug delivery system comprises from about 3 weight percent to about 97 weight percent of the at least one polyacrylic acid and from about 97 weight percent to about 3 weight percent of the at least one enteric polymer.

6. The controlled release drug delivery system of claim 4, wherein the at least one enteric polymer is selected from one or more polyacrylate copolymers, one or more cellulose derivatives, one or more polyvinyl acetate phthalates, shellac, or suitable combinations of two or more thereof.

7. A controlled release drug delivery system comprising:
from about 5 weight percent to about 95 weight percent of at least polyacrylic acid; and
from about 95 weight percent to about 5 weight percent of at least one enteric polymer,
wherein the controlled release drug delivery system is able to deliver in a controlled release manner at least one active pharmaceutical ingredient regardless of whether or not the at least one active pharmaceutical ingredient exhibits pH dependency.

8. The controlled release drug delivery system of claim 7, wherein the at least one enteric polymer is selected from one or more polyacrylate copolymers, one or more cellulose derivatives, one or more polyvinyl acetate phthalates, shellac, or suitable combinations of two or more thereof.

9. The controlled release drug delivery system of claim 7, wherein the at least one polyacrylic acid is a cross-linked polyacrylic acid selected from one or more carbomers, one or more polycarbophils, one or more copolymers of acrylic acid and alkyl acrylates, one or more copolymers of acrylic acid and alkyl vinyl ethers, copolymers of ethylene and maleic anhydride, one or more copolymers of maleic anhydride and alkyl vinyl ethers, or combinations of two or more thereof.

10. The controlled release drug delivery system of claim 7, wherein the delivery system can be used in conjunction with at least one active pharmaceutical ingredients selected from one or more cationic pharmaceutical compounds, one or more anionic pharmaceutical compounds, one or more amphoteric pharmaceutical compounds, one or more zwitterionic pharmaceutical compounds, one or more nonionic pharmaceutical compounds, or suitable combinations of two or more thereof.

11. The controlled release drug delivery system of claim 7, wherein the controlled release drug delivery system comprises from about 10 weight percent to about 85 weight percent of the at least one polyacrylic acid and from about 90 weight percent to about 15 weight percent of the at least one enteric polymer.

12. The controlled release drug delivery system of claim 11, wherein the at least one enteric polymer is selected from one or more polyacrylate copolymers, one or more cellulose derivatives, one or more polyvinyl acetate phthalates, shellac, or suitable combinations of two or more thereof.

13. A controlled release pharmaceutical composition comprising:
at least one active pharmaceutical ingredient; and
at least one controlled release drug delivery system, wherein the controlled release drug delivery system comprises:
at least one polyacrylic acid; and
at least one enteric polymer,
wherein the controlled release drug delivery system is able to deliver in a controlled release manner the at least one active pharmaceutical ingredient regardless of whether or not the at least one active pharmaceutical ingredient exhibits pH dependency.

14. The controlled release pharmaceutical composition of claim 13, wherein the at least one enteric polymer of the at least one controlled release drug delivery system, is selected from one or more polyacrylate copolymers, one or more cellulose derivatives, one or more polyvinyl acetate phthalates, shellac, or suitable combinations of two or more thereof.

15. The controlled release pharmaceutical composition of claim 13, wherein the at least one polyacrylic acid is a cross-linked polyacrylic acid selected from one or more carbomers, one or more polycarbophils, one or more copolymers of acrylic acid and alkyl acrylates, one or more copolymers of acrylic acid and alkyl vinyl ethers, copolymers of ethylene and maleic anhydride, one or more copolymers of maleic anhydride and alkyl vinyl ethers, or combinations of two or more thereof.

16. The controlled release pharmaceutical composition of claim 13, wherein the at least one active pharmaceutical ingredient is selected from one or more cationic pharmaceutical compounds, one or more anionic pharmaceutical compounds, one or more amphoteric pharmaceutical compounds, one or more zwitterionic pharmaceutical compounds, one or more nonionic pharmaceutical compounds, or suitable combinations of two or more thereof.

17. The controlled release pharmaceutical composition of claim 13, wherein the controlled release drug delivery system comprises from about 3 weight percent to about 97 weight percent of the at least one polyacrylic acid and from about 97 weight percent to about 3 weight percent of the at least one enteric polymer.

18. The controlled release pharmaceutical composition of claim 17, wherein the at least one enteric polymer is selected from one or more polyacrylate copolymers, one or more cellulose derivatives, one or more polyvinyl acetate phthalates, shellac, or suitable combinations of two or more thereof.

19. A controlled release pharmaceutical composition comprising:
at least one active pharmaceutical ingredient; and
at least one controlled release drug delivery system, wherein the controlled release drug delivery system comprises:
from about 5 weight percent to about 95 weight percent of at least polyacrylic acid; and
from about 95 weight percent to about 5 weight percent of at least one enteric polymer,
wherein the controlled release drug delivery system is able to deliver in a controlled release manner the at least one active pharmaceutical ingredient regardless of whether or not the at least one active pharmaceutical ingredient exhibits pH dependency.

20. The controlled release pharmaceutical composition of claim 19, wherein the at least one enteric polymer of the at least one controlled release drug delivery system, is selected from one or more polyacrylate copolymers, one or more cellulose derivatives, one or more polyvinyl acetate phthalates, shellac, or suitable combinations of two or more thereof.

21. The controlled release pharmaceutical composition of claim 19, wherein the at least one polyacrylic acid is a cross-linked polyacrylic acid selected from one or more carbomers, one or more polycarbophils, one or more copolymers of acrylic acid and alkyl acrylates, one or more copolymers of acrylic acid and alkyl vinyl ethers, copolymers of ethylene and maleic anhydride, one or more copolymers of maleic anhydride and alkyl vinyl ethers, or combinations of two or more thereof.

22. The controlled release pharmaceutical composition of claim 19, wherein the at least one active pharmaceutical ingredient is selected from one or more cationic pharmaceutical compounds, one or more anionic pharmaceutical compounds, one or more amphoteric pharmaceutical compounds, one or more zwitterionic pharmaceutical compounds, one or more nonionic pharmaceutical compounds, or suitable combinations of two or more thereof.

23. The controlled release pharmaceutical composition of claim 19, wherein the controlled release drug delivery system comprises from about 3 weight percent to about 97 weight percent of the at least one polyacrylic acid and from about 97 weight percent to about 3 weight percent of the at least one enteric polymer.

24. The controlled release pharmaceutical composition of claim 23, wherein the at least one enteric polymer is selected from one or more polyacrylate copolymers, one or more cellulose derivatives, one or more polyvinyl acetate phthalates, shellac, or suitable combinations of two or more thereof.
